(19) United States
(12) Patent Application Publication
(10) Pub. No.: US 2024/0225439 A1
(43) Pub. Date: Jul. 11, 2024

(54) METHOD AND APPARATUS FOR POSITIONING A TORIC INTRAOCULAR LENS

(71) Applicant: Munich Surgical Imaging GmbH, München (DE)

(72) Inventors: Ralf ENGELHARDT, Lübeck (DE); Benedikt WURM, München (DE)

(73) Assignee: Munich Surgical Imaging GmbH, München (DE)

(21) Appl. No.: 18/562,390

(22) PCT Filed: Mar. 22, 2022
(86) PCT No.: PCT/EP2022/057477

§ 371 Date: Nov. 20, 2023

(30) Foreign Application Priority Data
May 20, 2021 (DE) 10 2021 002 638.1

(51) Int. Cl. A61B 3/036; A61B 3/00; A61B 3/10; A61B 3/13

(52) U.S. Cl. CPC A61B 3/036; A61B 3/0033; A61B 3/0041; A61B 3/102; A61B 3/13

(57) ABSTRACT

The invention relates to a method for monitoring and/or evaluating the topographical positioning of a toric intraocular lens (14) during an operation on a human eye, by means of which the toric intraocular lens (14) is inserted into the human eye and is intended to be brought into its desired end position, the method comprising the following steps: determining at least one axis (7) of an astigmatism to be corrected of a cornea (1) of a human eye, in particular the axis (7) of the plane (2) of incidence of the strongest light refraction and/or the axis of the plane (3) of incidence of the weakest light refraction; and presenting the target axis (18) for the preferred axis (17) of the toric intraocular lens (14) relative to the determined axis (7) of the astigmatism in an electronic display device (19) of an apparatus (21), such that the movable toric intraocular lens (14) can be topographically situated and brought into its desired end position on the basis of the presentation. The method solves the problem of correctly situating and orienting a toric intraocular lens relative to a light-refracting object. The problem is also solved by an apparatus (21) for carrying out the method.

The invention relates to a method and an apparatus as claimed in the independent claims.

A position ascertaining arrangement for an intraocular lens is known from DE 10 2016 105 962 A1.

After the removal of the natural lens of a human eye, artificial lenses are often fitted into the eye. An artificial lens may, however, also be fitted into an eye in addition to the natural lens. Against this background, as artificial lenses, so-called intraocular lenses (abbreviated to IOL) which can be fitted into the human eye have become known.

There are very many intraocular lenses with different optical properties.

When fitting a so-called toric intraocular lens in order to correct human corneal astigmatism, it is necessary to provide the operating person with assistance for the alignment of a privileged axis of the intraocular lens.

A corneal distortion is referred to as corneal astigmatism. Specifically, this describes a defect in the refracting behavior of the human eye. The more curved the corneal surface is, the greater the light refraction is, but when there is a corneal astigmatism the corneal surface is curved to different extents in different regions so that the cornea has incidence planes with different light-refracting behavior.

Such an incidence plane is formed by a light ray incident parallel to the optical axis of the human eye with the optical axis itself. The axis of a corneal astigmatism may be defined by using an angle that an incidence plane makes with a plane, in particular a horizontal plane. Sometimes, the angle itself is also referred to as an axis.

When there is a corneal astigmatism, the human eye may have a first incidence plane for light rays which experience the greatest refracting power and a second incidence plane for light rays which experience the least refracting power. The fact that the light rays refracted differently in this way are focused not onto a single focal point on the retina but at different focal points in front of or behind the retina causes the formation of focal lines which lead to rodlike instead of pointlike vision.

Against this background, it is known that a toric intraocular lens has two different focal lengths in two mutually orthogonally oriented directions. Usually, such a lens has a first toric lens surface in the shape of a “cap” of a torus. Another lens surface is usually spherical. A toric intraocular lens is usually provided by the manufacturer with markings for a selected axis, namely a privileged axis.

Such a privileged axis may be associated with a strong or sharp corneal curvature, which is referred to in brief by a person skilled in the art as “steep”, or a shallow or level corneal curvature, which is referred to by a person skilled in the art as “flat”.

The optimal alignment of the privileged axis of the intraocular lens will have been calculated before the actual operation, usually with diagnostic equipment and special formulae. A measurement of the cornea, for example its diameter and particularly its astigmatism, may be carried out by means of an ophthalmometer. Such a measurement is called keratometry. So-called topometry allows measurement of the radii of curvature of the cornea.

By the aforementioned measurements, the profile of the surface of the cornea may be recorded. In particular, the structure of an intraocular lens to be fitted into the human eye may be calculated or ascertained. The axis of the corneal astigmatism to be corrected is usually also known from a preoperative diagnosis.

Both axes, the target axis of the privileged axis of the toric intraocular lens and the axis of the corneal astigmatism to be corrected, are usually specified relative to a vertical or horizontal plane of the eye.

Particularly during an operation on the living human eye, but also during work on inanimate optical systems, it is necessary to position the intraocular lens correctly.

Against this background, the object of the invention is to arrange and align a toric intraocular lens correctly relative to a light-refracting object.

The present invention achieves the aforementioned object by the features of the independent claims.

According to the invention, it has been found that the target axis for the privileged axis of the toric intraocular lens, that is to say the alignment of the privileged axis relative to the ascertained axis of the astigmatism to be corrected, may be represented online in an electronic display device of an apparatus, that is to say simultaneously with the movement of the intraocular lens, so that the mobile intraocular lens can be arranged in the correct position and brought into its intended final location with the aid of the simultaneous representation, in particular during the operation on the human eye.

It is not necessary to use previously stored or prepared reference images or registering images during the operation, but merely for the preoperatively determined angular separation of the target axis from the axis of the corneal astigmatism to be known. The currently existing state of the eye may be used in order to ascertain the target axis. Before or at the start of the operation, that is to say before the access incisions are opened, the axis of the corneal astigmatism may be determined relative to structures of the eye which are visible in the microscope image and are not altered during the operation. Since the target axis is known relative to this axis from preliminary examinations and preliminary calculations, the target axis may subsequently be represented visually at any time, for example during the operation, in order to align the privileged axis of the IOL accordingly.

In order to ascertain the target axis, the center of the limbus and/or the vertex of the cornea could be ascertained, the center of the limbus and/or the vertex being used as a common point of rotation and/or apex for the target axis and for the axis of the astigmatism. Besides the lens and the cornea, the human eye has a so-called limbus, the medical term for which is limbus corneae. The limbus is a transition region between the cornea and the sclera of the human eye. The so-called vertex is the point of intersection of the surface of the cornea facing toward an incident light ray with the optical axis. In order to visualize the target axis suitably, registering of the vertex in a camera image may be performed, and live or simultaneous determination of the vertex or determination of the center of the limbus may be performed while carrying out the method described here. What is important is that all axes that are set in relation to one another have the same reference point or point of rotation, about which they turn, so that the location of the axes relative to one another is clearly representable.

The display of the target axis could be updated according to predetermined time intervals during a predeterminable period of time, so that the instantaneous and correct location of the target axis is constantly displayed, in particular during an operation on the human eye. In this way, the correct location of the target axis is constantly displayed. Influences due to the operation may be compensated for computationally so that the correct target axis is constantly displayed.

Against this background, movements of the human eye may be recorded and the updating of the target axis may be carried out in such a way that the target axis is tracked to the eye movements. Preferably, the movement of the eye is calculated with the aid of the position of vessels in the sclera. It is, however, also possible to record the movement by using iris recognition.

It could be possible that that no preoperatively prepared reference image or registering image, in which the target axis is displayed relative to an axis of the astigmatism, is used. This ensures that the current state of the eye to be treated is used as a basis for the ascertaining of the target axis. Alternatively or additionally, it could be possible that the location or orientation of the target axis relative to the axis of the astigmatism has not been ascertained before the ascertaining of the axis of the astigmatism. The location of the target axis relative to the axis of the astigmatism could be calculated and displayed only after the preoperative ascertaining of the at least one axis of the astigmatism.

At least one orientation means for arranging the intraocular lens or the privileged axis of the toric intraocular lens in the correct position could be displayed by means of the display device. Thus, the operating person may better assess whether the toric intraocular lens is correctly positioned. The operating person is less restricted in their treatment and diagnosis to estimates or empirical values, but may very accurately read angles and inclinations of the intraocular lens, preferably with an error tolerance of 1-2 degrees. It is therefore conceivable for an angle scale to be overlaid in the display device.

The orientation means could therefore have at least one line and/or angle graduation. A line in interrupted or continuous form may be used in order to bring the usually linear markings of the toric intraocular lens, which define the privileged axis to be aligned, into correspondence with the target axis. The target axis is to this extent formed by one or more lines of the visually overlaid orientation means.

The position and dimension of cuts or incisions to be performed may be displayed on the display device. The operating person is thereby additionally assisted.

An apparatus for carrying out a method of the type described here, or for carrying out individual steps of the method described here, comprises an electronic display device for displaying the target axis for the privileged axis of a toric intraocular lens. The display device is preferably configured as a screen. The apparatus optionally further comprises a calculation unit, the calculation unit receiving data from which the calculation unit can calculate the location of the target axis in order to visualize it on the display device. The apparatus alternatively or additionally comprises a control unit, the control unit being manually controllable, in order to visualize the location of the target axis on the display device. A preoperatively measured axis of the corneal astigmatism and the target axis for the toric intraocular lens may thus be delivered to the calculation unit. The delivery is carried out by using a suitable interface. Alternatively, these data may be entered into a menu of a user interface by a user. Since the location of the relevant axes relative to one another is thereby known, the calculation unit can calculate and suitably visualize the location of the target axis. This visualization may also be used by the operating person for manual marking of the target axis.

The apparatus could be configured as an operation microscope or be characterized by its being coupled to an operation microscope or integrated into an operation microscope. The operation microscope could comprise a camera and/or a device for carrying out optical coherence tomography and/or a device for carrying out reflection illuminations.

The determination of the axis of the corneal astigmatism may be carried out by using reflection illuminations installed in an operation microscope, in particular by the keratometer method. Alternatively or additionally, the determination of the axis of the corneal astigmatism could be carried out by other suitable means, for example by using an integrated device for carrying out optical coherence tomography (OCT).

The axis thereby preoperatively measured of the corneal astigmatism and the target axis for the toric intraocular lens could be delivered to a calculation unit connected to the operation microscope. The delivery is either carried out by using a suitable interface or is entered into a menu of a user interface by the user. Since the location of the axes relative to one another is thereby known, the calculation unit can calculate and suitably visualize the location of the target axis. The visualization may also be used by the operating person for manual marking of the target axis.

If the operation microscope is equipped with a suitable camera, the calculation unit may calculate the location of the target axis in an acquired camera image. In the further course of the operation, the movement of the eye may be registered with the aid of the current camera image by the calculation unit using suitable image processing and the respective current target axis may be visualized in a suitable way.

In this case, the need for manual registering is obviated. The visualization also continues to be possible during the operative intervention, even when the current axis of the corneal astigmatism is modified relative to the preoperative location by manipulations, for example incisions.

Against this background, the compilation of an individual registering image for astigmatism may be envisioned. From a preliminary examination, the target angle for an intraocular lens relative to a steep axis of the cornea is known. At the start of the operation, the steep axis on the cornea is determined with a keratometer illumination, the eye not yet being treated at this time, and stored together with a registering image which contains the vessels of the sclera. From these data, the target axis for the intraocular lens may be calculated and displayed online at any time.

### IN THE DRAWINGS

FIG. 1 shows a schematic representation of the corneal astigmatism of a human eye with the aid of the light-refracting behavior of the cornea,

FIG. 2 schematically shows a toric intraocular lens, the privileged axis of which is oriented so as to coincide with the axis of an incidence plane, namely the axis of the corneal astigmatism to be corrected, and

FIG. 3 shows at the top an enlarged view of a schematic representation of an electronic display device which permanently displays the correct target axis of the toric intraocular lens to an operating person during the operation, independently of possible eye movements of the patient, and schematically shows at the bottom an apparatus having the display device, further components of the apparatus being schematically represented.

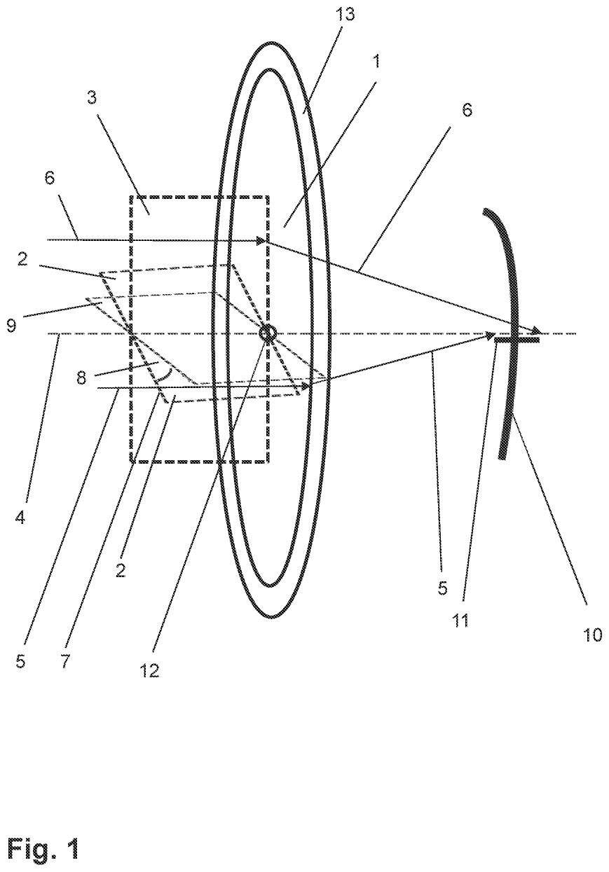

FIG. 1 schematically shows a defect in the refracting behavior of the cornea 1 of the human eye, namely a corneal astigmatism. The more curved the corneal surface is, the greater the light refraction is, but when there is a corneal astigmatism the corneal surface is curved to different extents in different regions so that the cornea has incidence planes 2, 3 with different refracting behavior.

An incidence plane 2, 3 is formed by a light ray 5, 6 incident parallel to the optical axis 4 of the human eye with the optical axis 4 itself. To this extent, the first light ray 5 lies in the first incidence plane 2 and the second light ray 6 lies in the second incidence plane 3, both light rays being parallel to the optical axis 4. The axis 7 of a corneal astigmatism to be corrected may be defined by using an angle 8 that a first incidence plane 2 makes with the horizontal plane 9. Sometimes, the angle 8 itself is also referred to as an axis.

When there is a corneal astigmatism, the human eye may have the first incidence plane 2 for first light rays 5 which experience the greatest refracting power and a second incidence plane 3 for second light rays 6 which experience the least refracting power.

The fact that the light rays 5, 6 refracted differently in this way are focused not onto a single focal point on the retina 10 but at different focal points in front of or behind the retina 10 causes the formation of focal lines 11 which lead to rodlike instead of pointlike vision.

The optical axis 4 passes through the vertex 12 of the cornea 1. The vertex 12 coincides with the center of the limbus 13.

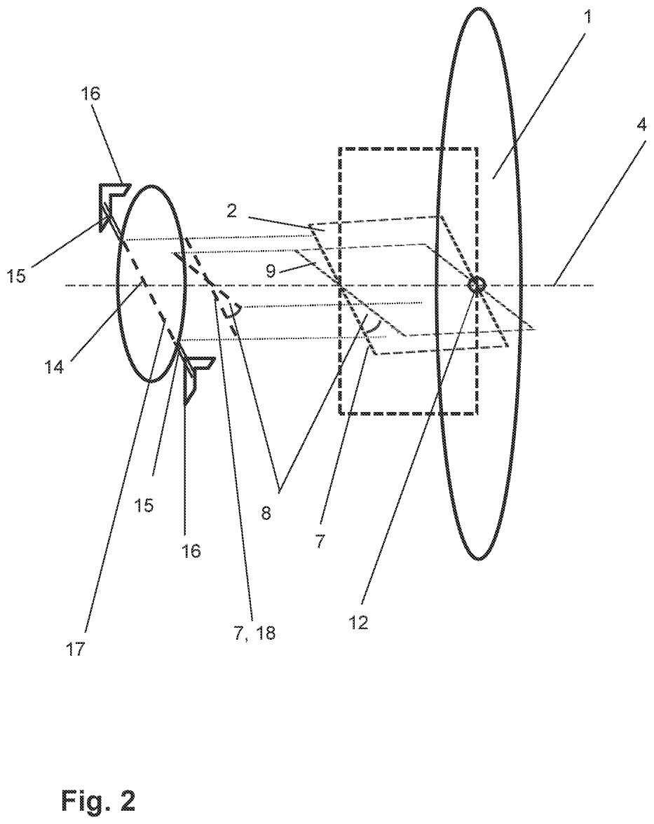

FIG. 2 shows a toric intraocular lens 14, which is provided by the manufacturer with linear markings 15 in the region of the haptics 16. The linear markings 15 define a selected axis, namely a privileged axis 17. The privileged axis 17 is associated here with a strong corneal curvature, which is referred to in brief by a person skilled in the art as “steep”. The axis 7 of the corneal astigmatism to be corrected is also the target axis 18 for the intraocular lens 14. This means that the privileged axis 17 specified by markings 15 must be brought into correspondence with the axis 7 of the astigmatism to be corrected, in order to operatively eliminate the corneal astigmatism.

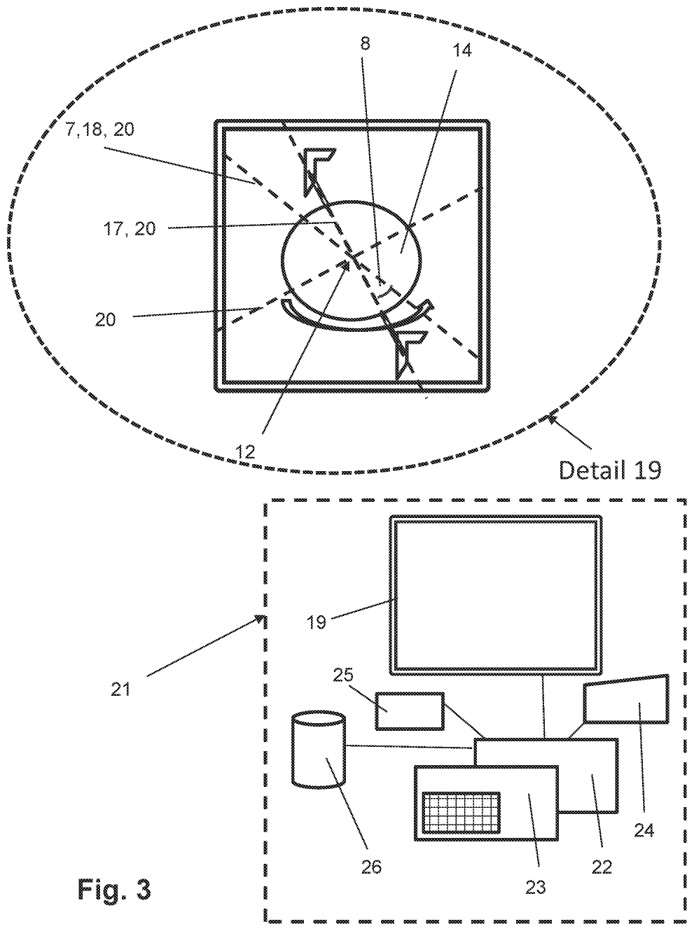

FIG. 3 schematically shows a method for monitoring the correct positioning of a toric intraocular lens 14 during an operation on the human eye, by which the toric intraocular lens 14 is to be fitted into the human eye and brought into its intended final location.

The method may comprise the following steps:
ascertaining at least one axis 7 of the astigmatism to be corrected of the cornea 1 of the human eye according to FIGS. 1 and 2, namely the axis 7 of the incidence plane 2 of the strongest light refraction,
ascertaining the target axis 18, the relative location of which with respect to an axis 7 of the corneal astigmatism to be corrected is known from preliminary examinations and preliminary calculations,
storing the target axis 18 and/or its location relative to structures of the eye which are visible in an image, in particular a microscope image, and are not altered during the operation,
permanently representing the target axis 18 for the privileged axis 17 of the toric intraocular lens 14 relative to the ascertained axis 7 of the astigmatism in an electronic display device 19 of an apparatus 21, so that the mobile toric intraocular lens 14 can be arranged in the correct position and brought into its intended final location with the aid of the representation.

The display device 19 is a screen.

In order to ascertain the target axis 18, the center of the limbus and/or the vertex 12 of the cornea 1 is ascertained, the center of the limbus and/or the vertex 12 being used as a common point of rotation and/or apex for the target axis 18 and for the axis 7 of the astigmatism, as well as for the privileged axis 17.

It may be seen that the toric intraocular lens 14 and its privileged axis 17 need to be rotated by the angle 8 about the vertex 12, which corresponds to the center or the optical axis of the toric intraocular lens 14, until the privileged axis 17 lies on the target axis 18 and then coincides with the target axis 18.

The display of the target axis 18 is updated according to predeterminable time intervals during a predeterminable period of time, so that the instantaneous and correct location of the target axis 18 is permanently displayed, in particular during an operation on the human eye.

The movements of the human eye are recorded and the updating of the target axis 18 is carried out in such a way that the target axis 18 is tracked to the eye movements.

No preoperatively prepared reference image or registering image, in which the target axis 18 is displayed relative to the axis 7 of the astigmatism, is used.

The location of the target axis 18 relative to the axis 7 of the astigmatism is not ascertained before the ascertaining of the axis 7 of the astigmatism. The location of the target axis 18 relative to the axis 7 of the astigmatism is calculated and displayed after the preoperative ascertaining of the axis 7 of the astigmatism.

At least one orientation means 20 for arrangement of the toric intraocular lens 14 or the privileged axis 18 of the toric intraocular lens 14 in the correct position is displayed by means of the display device 19.

All axes 7, 17, 18 may be electronically represented by orientation means 20, in particular by lines. An orientation means 20 has at least one line and/or angle graduation.

FIG. 3 shows an apparatus 21 for carrying out the method described above, which comprises the electronic display device 19 for displaying the target axis 18 for a privileged axis 17 of a toric intraocular lens 14.

The apparatus 21 further has a calculation unit 22 and a control unit 23, the calculation unit 22 receiving data from which the calculation unit 22 can calculate the location of the target axis 18 in order to visualize it on the display device 19, and the control unit 23 being manually controllable in order to visualize the location of the target axis 18 on the display device 19.

The apparatus 21 is an operation microscope, the operation microscope comprising a camera 24 and a device 25 for carrying out optical coherence tomography (OCT) and a device 26 for carrying out reflection illuminations.

### LIST OF REFERENCE SIGNS

1 cornea
2 first incidence plane
3 second incidence plane
4 optical axis
5 first light ray in first incidence plane
6 second light ray in second incidence plane
7 axis to be corrected of the corneal astigmatism
8 angle of 2 with horizontal plate
9 horizontal plane
10 retina
11 focal line
12 vertex or center of the limbus
13 limbus
14 toric intraocular lens
15 marking for privileged axis
16 haptic of 14
17 privileged axis of 14
18 target axis of 14
19 display device
20 orientation means in 19
21 apparatus or operation microscope
22 calculation unit
23 control unit
24 camera
25 device for carrying out optical coherence tomography
26 device for carrying out reflection illuminations

1. A method for monitoring and/or evaluating the correct positioning of a toric intraocular lens during an operation on the human eye, by which the toric intraocular lens is to be fitted into the human eye and brought into its intended final location, comprising the following steps:
ascertaining at least one axis of an astigmatism to be corrected of a cornea of a human eye, in particular the axis of the incidence plane of the strongest light refraction and/or the axis of the incidence plane of the weakest light refraction, and
representing the target axis for the privileged axis of the toric intraocular lens relative to the ascertained axis of the astigmatism in an electronic display device of an apparatus, so that the mobile toric intraocular lens can be arranged in the correct position and brought into its intended final location with the aid of the representation.

2. The method as claimed in claim 1, wherein in order to ascertain the target axis the center of the limbus and/or the vertex of the cornea is ascertained, the center of the limbus and/or the vertex being used as a common point of rotation and/or apex for the target axis and for the axis of the astigmatism.

3. The method as claimed in claim 1, wherein the display of the target axis is updated according to predeterminable time intervals during a predeterminable period of time, so that the instantaneous and correct location of the target axis is constantly displayed, in particular during an operation on the human eye.

4. The method as claimed in claim 3, wherein the movements of the human eye are recorded and the updating of the target axis is carried out in such a way that the target axis is tracked to the eye movements.

5. The method as claimed in claim 1, wherein no preoperatively prepared reference image or registering image, in which the target axis is displayed relative to an axis of the astigmatism, is used, and/or in that the location of the target axis relative to the axis of the astigmatism has not been ascertained before the ascertaining of the axis of the astigmatism, but rather the location of the target axis relative to the axis of the astigmatism is calculated and displayed after the preoperative ascertaining of the at least one axis of the astigmatism.

6. The method as claimed in claim 1, wherein at least one orientation means for arrangement of the toric intraocular lens or the privileged axis of the toric intraocular lens in the correct position is displayed by means of the display device.

7. The method as claimed in claim 1, wherein the privileged axis of the toric intraocular lens is displayed by means of the display device by at least one orientation means, the position of the privileged axis of the toric intraocular lens being established by the recognition of markings on the toric intraocular lens.

8. The method as claimed in claim 6, wherein the orientation means has at least one line and/or angle graduation.

9. The method as claimed in claim 1, wherein the position and dimension of the cuts or incisions to be performed are displayed on the display device.

10. An apparatus for carrying out a method as claimed in claim 1, comprising an electronic display device for displaying the target axis for a privileged axis of a toric intraocular lens, wherein the apparatus further comprises a calculation unit and/or a control unit, wherein the calculation unit receives data from which the calculation unit can calculate the location of the target axis in order to visualize it on the display device, and wherein the control unit is manually controllable in order to visualize the location of the target axis on the display device.

11. The apparatus as claimed in claim 10, characterized by its being configured as an operation microscope or by its being coupled to an operation microscope or integrated into an operation microscope, the operation microscope comprising a camera and/or a device for carrying out optical coherence tomography and/or a device for carrying out reflection illuminations.
